SEQUENCE LISTING (2 sequences)

SEQ ID NO: 1 - protein, 44 aa, Artificial
  AVE0010 (44 AS) Position 44 (Lys) is amidated
HGEGTFTSDLSKQMEEEAVRLFLEWLKNGGPSSGAPPSKKKKKK

SEQ ID NO: 2 - protein, 39 aa, Artificial
  Heloderma Suspectum Position 39 (Ser) is amidated
HGEGTFTSDLSKQMEEEAVRLFIEWLKNGGPSSGAPPPS

(19) United States
(12) Patent Application Publication
(10) Pub. No.: US 2011/0118178 A1
(43) Pub. Date: May 19, 2011

(54) METHOD OF TREATMENT OF DIABETES TYPE 2 COMPRISING ADD-ON THERAPY TO INSULIN GLARGINE AND METFORMIN

(75) Inventors: Louise SILVESTRE, Paris (FR); Elisabeth SOUHAMI, Les Pavillons sous Bois (FR); Xiaodan WEI, Bridgewater, NJ (US)

(73) Assignee: SANOFI-AVENTIS DEUTSCHLAND GMBH, Frankfurt am Main (DE)

(21) Appl. No.: 12/617,805

(22) Filed: Nov. 13, 2009

(51) Int. Cl. A61K 38/28; A61P 3/10

(52) U.S. Cl. 514/5.9; 514/6.9; 514/6.5

(57) ABSTRACT

A method for the treatment of diabetes mellitus type 2 comprising administering
(d) desPro³⁶Exendin-4(1-39)-Lys₆-NH₂ or/and a pharmaceutically acceptable salt thereof,
(e) insulin glargine or/and a pharmaceutically acceptable salt thereof, and
(f) metformin or/and a pharmaceutically acceptable salt thereof,
to a subject in need thereof.

Subject of the present invention is a method for treatment of diabetes type 2 with AVE0010 (lixisenatide) as add-on therapy to administration of insulin glargine and metformin.

Metformin is a biguanide hypoglycemic agent used in the treatment of Type 2 diabetes mellitus not responding to dietary modification. Metformin improves glycemic control by improving insulin sensitivity. Metformin is usually administered orally.

Insulin is a polypeptide having 51 amino acid residues. Insulin consists of the A chain having 21 amino acid residues, and the B chain having 30 amino acid residues. The chains are coupled by 2 disulfide bridges. Insulin formulations have been used for a long time for therapy of diabetes mellitus type 1 and 2. Recently, insulin derivatives and insulin analogues have been used.

However, control diabetes mellitus type 2 by metformin and insulin may be insufficient. Thus, in these patients, additional measures for controlling diabetes mellitus type 2 may be required.

A first aspect of the present invention is a method for the treatment of diabetes mellitus type 2 comprising administering
(a) desPro³⁶Exendin-4(1-39)-Lys₆-NH₂ (AVE0010, lixisenatide) or/and a pharmaceutically acceptable salt thereof,
(b) insulin glargine or/and a pharmaceutically acceptable salt thereof, and
(c) metformin or/and a pharmaceutically acceptable salt thereof,
   to a subject in need thereof.

The compounds of (a), (b) and (c) may be administered to a subject in need thereof, in an amount sufficient to induce a therapeutic effect.

The compound desPro³⁶Exendin-4(1-39)-Lys₆-NH₂ (AVE0010, lixisenatide) is a derivative of Exendin-4. AVE0010 is disclosed as SEQ ID NO:93 in WO 01/04156:

SEQ ID NO: 1 AVE0010 (44 AS)
H-G-E-G-T-F-T-S-D-L-S-K-Q-M-E-E-E-A-V-R-L-F-I-E-W-
L-K-N-G-G-P-S-S-G-A-P-P-S-K-K-K-K-K-K-NH₂
SEQ ID NO: 2 Exendin-4 (39 AS)
H-G-E-G-T-F-T-S-D-L-S-K-Q-M-E-E-E-A-V-R-L-F-I-E-W-
L-K-N-G-G-P-S-S-G-A-P-P-P-S-NH₂

Exendins are a group of peptides which can lower blood glucose concentration. The Exendin analogue AVE0010 is characterised by C-terminal truncation of the native Exendin-4 sequence. AVE0010 comprises six C-terminal lysine residues not present in Exendin-4.

In the context of the present invention, AVE0010 includes pharmaceutically acceptable salts thereof. The person skilled in the art knows pharmaceutically acceptable salts of AVE0010. A preferred pharmaceutically acceptable salt of AVE0010 employed in the present invention is acetate.

AVE0010 (desPro³⁶Exendin-4(1-39)-Lys₆-NH₂) or/and a pharmaceutically acceptable salt thereof may be administered parenterally, e.g. by subcutaneous injection. Suitable injection devices, for instance the so-called “pens” comprising a cartridge comprising the active ingredient, and an injection needle, are known. AVE0010 or/and a pharmaceutically acceptable salt thereof may be administered in a suitable amount, for instance in an amount in the range of 10 to 15 μg per dose or 15 to 20 μg per dose once a day (progressive titration from 10 to 15 and to 20 μg/day. 20 μg is the effective maintenance dose).

In the present invention, AVE0010 or/and a pharmaceutically acceptable salt thereof may be administered in a daily dose in the range of 10 to 15 μg or in the range of 15 to 20 μg (progressive titration from 10 to 15 and to 20 μg/day. 20 μg is the effective maintenance dose). AVE0010 or/and a pharmaceutically acceptable salt thereof may be administered by one injection per day.

Insulin glargine (Lantus) is Gly(A21)-Arg(B31)-Arg(B32)-human insulin. In the context of the present invention, insulin glargine includes pharmaceutically acceptable salts thereof.

Insulin glargine or/and a pharmaceutically acceptable salt thereof may be administered by injection (by subcutaneous injection). Suitable injection devices, for instance the so-called “pens” comprising a cartridge comprising the active ingredient, and an injection needle, are known. Insulin glargine or/and a pharmaceutically acceptable salt thereof may be administered in a suitable amount, for instance in an amount of at least 10 units per day (the initial dose is 10 units; 80 units is the maximal dose possible with the pen with 1 injection)

In the present invention, insulin glargine or/and a pharmaceutically acceptable salt thereof may be administered in a daily dose of at least 10 units. Insulin glargine or/and a pharmaceutically acceptable salt thereof may be administered by one injection per day.

In the present invention, AVE0010 or/and a pharmaceutically acceptable salt thereof may be provided in a liquid composition The skilled person knows liquid compositions of AVE0010 suitable for subcutaneous administration.

In the present invention, insulin glargine or/and a pharmaceutically acceptable salt thereof may be provided in a liquid composition The skilled person knows liquid compositions of insulin glargine suitable for subcutaneous administration.

A liquid composition employed herein may have an acidic or a physiologic pH. An acidic pH preferably is in the range of pH 1-6.8, pH 3.5-6.8, or pH 3.5-5. A physiologic pH preferably is in the range of pH 2.5-8.5, pH 4.0 to 8.5, or pH 6.0 to 8.5. The pH may be adjusted by a pharmaceutically acceptable diluted acid (typically HCl) or pharmaceutically acceptable diluted base (typically NaOH).

The preferred pH is in the range of pH 3.5 to 5.0.

The liquid composition may contain a buffer, such as a phosphate, a citrate, an acetate. Preferably, it can contain an acetate buffer, in quantities up to 5 μg/mL, up to 4 μg/mL or up to 2 μg/mL.

The liquid composition employed herein may comprise a suitable preservative. A suitable preservative may be selected from phenol, m-cresol, benzyl alcohol and p-hydroxybenzoic acid ester. A preferred preservative is m-cresol. However, the preferred liquid composition does not contain a preservative.

The liquid composition employed herein may comprise a tonicity agent. A suitable tonicity agent may be selected from glycerol, lactose, sorbitol, mannitol, glucose, NaCl, calcium or magnesium containing compounds such as CaCl₂. The concentration of glycerol, lactose, sorbitol, mannitol and glucose may be in the range of 100-250 mM. The concentration of NaCl may be up to 150 mM. A preferred tonicity agent is glycerol.

In addition, the liquid composition may contain L-methionin from 0.5 μg/mL to 20 μg/mL, preferably from 1 μg/mL to 5 μg/mL. Preferably it contains L-methionin.

Metformin is the international nonproprietary name of 1,1-dimethylbiguanide (CAS Number 657-24-9). In the present invention, the term “metformin” includes any pharmaceutically acceptable salt thereof.

In the present invention, metformin may be administered orally. The skilled person knows formulations of metformin suitable for treatment of diabetes type 2 by oral administration. Metformin may be administered in a dose of at least 1.5 g/day. For oral administration, metformin may be formulated in a solid dosage form, such as a tablet or pill.

In the present invention, desPro³⁶Exendin-4(1-39)-Lys₆-NH₂ or/and a pharmaceutically acceptable salt is administered in an add-on therapy to administration of metformin and insulin glargine.

In the present invention, the terms “add-on”, “add-on treatment” and “add-on therapy” relate to treatment of diabetes mellitus type 2 with metformin, insulin glargine and AVE0010. Metformin, insulin glargine and AVE0010 may be administered within a time interval of 24 h. Metformin, insulin glargine and AVE0010 each may be administered in a once-a-day-dosage. Metformin may be administered by a different administration route than insulin glargine and AVE0010. Metformin may be administered orally, whereas AVE0010 and insulin glargine may be administered subcutaneously.

The subject to be treated by the method of the present invention may have a fasting plasma glucose concentration of at least 7 mmol/L or/and 2 hours postprandial plasma glucose of at least 11.1 mmol/L. The subject may have a HbA1c value in the range of 7% to 10%.

The subject to be treated by the method of the present invention may be an adult subject. The subject may have an age in the range of 18 to 50 years.

The method of the present invention preferably is a method of treatment of a subject suffering from diabetes type 2, wherein diabetes type 2 is not adequately controlled by treatment with metformin and insulin alone, for instance with a dose of at least 1.5 g/day metformin and a dose of insulin of at least 10 units, preferably of 15 to 80 U/day for 3 months.

Another aspect of the present invention is a pharmaceutical combination comprising
(a) desPro³⁶Exendin-4(1-39)-Lys₆-NH₂ or/and a pharmaceutically acceptable salt thereof,
(b) insulin glargine or/and a pharmaceutically acceptable salt thereof, and
(c) metformin or/and a pharmaceutically acceptable salt thereof.

Preferably, the combination of the present invention is for treatment of diabetes mellitus type 2.

The combination of the present invention may be administered as described herein in the context of the method of the present invention. The compounds (a), (b) and (c) of the combination of the present invention may be formulated as described herein in the context of the method of the present invention.

Yet another aspect of the present invention is the use of a combination comprising
(a) desPro³⁶Exendin-4(1-39)-Lys₆-NH₂ or/and a pharmaceutically acceptable salt thereof,
(b) insulin glargine or/and a pharmaceutically acceptable salt thereof, and
(c) metformin or/and a pharmaceutically acceptable salt thereof,
   for the production of a medicament for the treatment of diabetes mellitus type 2.

The medicament comprises desPro³⁶Exendin-4(1-39)-Lys₆-NH₂, insulin glargine and metformin in separate formulations, as described herein.

The invention is further illustrated by the following example.

### EXAMPLE

24-week treatment of diabetes type 2 with lixisenatide (AVE0010) as add-on therapy to insulin glargine and metformin

Subject of the example is a randomized, placebo-controlled, 2-arm parallel-group, multicenter study with a 24-week double-blind treatment period assessing the efficacy and safety of Lixisenatide in patients with type 2 diabetes insufficiently controlled with insulin glargine and metformin.

### Study Primary Objectives

The primary objective of this study is to assess the effects on glycemic control of lixisenatide in comparison to placebo as an add-on treatment to insulin glargine and metformin over a period of 24 weeks.

### Study Secondary Objectives

The secondary objectives are:
To assess the effects of lixisenatide (AVE0010) on the percentage of patients reaching HbA1c<7% and < or =6.5%, on plasma glucose (fasting, post-prandial during a standardized meal challenge test, 7-point self monitored profiles), body weight, insulin glargine doses.
To evaluate lixisenatide safety and tolerability as add on treatment to insulin glargine and metformin.
To assess the impact of lixisenatide on treatment satisfaction using the Diabetes Treatment Satisfaction Questionnaire (state) (DTSQs) in the participating countries where it is validated.

| Condition | Intervention | Phase |
|---|---|---|
| Type 2 Diabetes Mellitus | Drug: lixisenatide (AVE0010) | Phase III |
| | Drug: placebo | |
| | Drug: insulin glargine (HOE901) | |

Study Type: Interventional
Study Design: Treatment, Randomized, Double Blind (Subject, Caregiver, Investigator, Outcomes Assessor), Placebo Control, Parallel Assignment, Efficacy Study

### Primary Outcome Measures:

Change in glycated hemoglobin (HbA1c) (time frame: 24 weeks, designated as safety issue: no)

### Secondary Outcome Measures:

Percentage of patients with HbA1c<7%, < or =6.5% (time frame: 24 weeks, designated as safety issue: no)
Change in postprandial plasma glucose (time frame: 24 weeks, designated as safety issue: no)
Change in fasting plasma glucose (time frame: 24 weeks, designated as safety issue: no)
Change in 7-point Self Monitored Plasma Glucose (SMPG) profiles (time frame: 24 weeks, designated as safety issue: no)
Change in body weight (time frame: 24 weeks, designated as safety issue: no)
Change in insulin glargine dose (time frame: 24 weeks, designated as safety issue: no)
Percentage of patients requiring rescue therapy during the double-blind period (time frame: 24 weeks, designated as safety issue: no)
Change in treatment satisfaction score (DTSQ questionnaire, time frame: 24 weeks, designated as safety issue: no)

### Estimated Enrolment: 290

| Arms | Assigned interventions |
|---|---|
| Lixisenatide: Experimental | Drug: lixisenatide (AVE0010) |
| 24-week treatment with lixisenatide once | solution for subcutaneous injection |
| daily on top of insulin glargine (both | Drug: insulin glargine (HOE901) |
| injected in the morning within 1 hour | solution for subcutaneous injection |
| prior to breakfast) and metformin (at | |
| least 1.5 g/day) | |
| Placebo: Placebo Comparator | Drug: placebo |
| 24-week treatment with placebo once | solution for subcutaneous injection |
| daily on top of insulin glargine (both | Drug: insulin glargine (HOE901) |
| injected in the morning within 1 hour | solution for subcutaneous injection |
| prior to breakfast) and metformin (at | |
| least 1.5 g/day) | |

### DETAILED DESCRIPTION

The study will comprise 3 periods:
An up-to 14-week screening period, which includes an up to 2-week screening phase and a 12-week run-in phase with introduction and titration of insulin glargine on top of metformin+/−TZDs.
At the end of the run-in phase, patients whose HbA1c (centralized assay) is > or =7% and < or =9% and whose mean fasting SMPG calculated from the self measurements for the 7 days prior to visit 12 (week-1) is less than or equal to 126 mg/dl (7.0 mmol/l), will enter a 24-week double-blind randomized treatment period comparing lixisenatide to placebo (on top of insulin glargine+metformin+/−TZDs).
A 3 day-safety follow up period.
   Maximum duration of 39 weeks±7 days

### Eligibility

Ages Eligible for Study: 18 Years and older

### Genders Eligible for Study: Both

### Accepts Healthy Volunteers: No

### Inclusion Criteria:

At screening
Patients with type 2 diabetes mellitus, as defined by WHO (fasting plasma glucose > or =7 mmol/L (126 mg/dL) or 2 hours postprandial plasma glucose > or =11.1 mmol/L (200 mg/dL), diagnosed at least 1 year before the screening visit
For at least 3 months: treatment with a stable dose of metformin > or =1.5 g/day or combination of stable doses of metformin > or =1.5 g/day with sulfonylureas (SUs) (to be stopped at visit 1) and/or Thiazolidinediones (TZDs)
Glycated hemoglobin (HbA1c) > or =7.0 and < or =10%
   At the end of the run in phase and before randomization:
HbA1c > or =7.0 and < or =9%
Mean fasting Self Monitored Plasma Glucose (SMPG) calculated from the self measurements for the 7 days prior to visit 12 (week-1) is less than or equal to 126 mg/dll (7.0 mmol/l)

### Exclusion Criteria:

At screening:
Pregnancy or lactation
Women of childbearing potential with no effective contraceptive method.
Type 1 diabetes mellitus
Metformin not at a stable dose of at least 1.5 g/day for at least 3 months prior to the screening visit.
Use of oral or injectable antidiabetic or hypoglycemic agents other than metformin, sulfonylurea and thiazolidinediones within 3 months prior to the time of screening, use of weight loss drugs if not at a stable dose for at least 3 months prior to the screening visit.
History of hypoglycemia unawareness.
History of unexplained pancreatitis, chronic pancreatitis, pancreatectomy, stomach/gastric surgery, inflammatory bowel disease
History of metabolic acidosis, including diabetic ketoacidosis within 1 year prior to screening
Hemoglobinopathy or hemolytic anemia, blood or plasma products transfusion within 3 months prior to the time of screening
Within the last 6 months prior to screening: history of myocardial infarction, stroke, or heart failure requiring hospitalization
Known history of drug or alcohol abuse within 6 months prior to the time of screening
Uncontrolled or inadequately controlled hypertension at the time of screening with a resting systolic or diastolic blood pressure >180 mmHg or >110 mmHg, respectively
Use of systemic glucocorticoids (excluding topical application or inhaled forms) for one week or more within 3 months prior to the time of screening
Use of any investigational drug within 3 months prior to screening
Renal impairment defined with serum creatinine >1.4 mg/dL in women and >1.5 mg/dL in men
History of hypersensitivity to insulin glargine or to any of the excipients
Clinically relevant history of gastrointestinal disease associated with prolonged nausea and vomiting, including (but not limited to): gastroparesis, unstable (i.e worsening) and not controlled (i.e prolonged nausea and vomiting) gastroesophageal reflux disease requiring medical treatment, within 6 months prior to the time of screening
Any previous treatment with lixisenatide (e.g. participation in a previous study with lixisenatide)
Allergic reaction to any GLP-1 receptor agonist in the past (e.g. exenatide, liraglutide) or to metacresol

Additional Exclusion Criteria During or at the End of the Run-in Phase Before Randomization:
Informed consent withdrawal (patient who is not willing to continue or fails to return)
Mean fasting SMPG calculated from the self-measurements for the 7 days prior to visit 12 (week-1) is >126 mg/dl (7.0 mmol/l)
HbA1c measured at visit 12 (week-1) is <7% or >9%,
Amylase and/or lipase >3 times the upper limit of the normal laboratory range at visit 12 (week-1)

The above information is not intended to contain all considerations relevant to a patient's potential participation in a clinical trial.

We claim:

1. A method for treating diabetes mellitus type 2 comprising administering
(a) desPro³⁶Exendin-4(1-39)-Lys₆-NH₂ or a pharmaceutically acceptable salt thereof,
(b) insulin glargine or a pharmaceutically acceptable salt thereof, and
(c) metformin or a pharmaceutically acceptable salt thereof,
to a subject in need thereof.

2. The method according to claim 1, wherein desPro³⁶Exendin-4(1-39)-Lys₆-NH₂ or a pharmaceutically acceptable salt thereof is administered subcutaneously.

3. The method according to claim 1, wherein insulin glargine or a pharmaceutically acceptable salt thereof is administered subcutaneously.

4. The method according to claim 1, wherein the metformin is administered orally.

5. The method according to claim 1, wherein desPro³⁶Exendin-4(1-39)-Lys₆-NH₂ or a pharmaceutically acceptable salt is administered in an add-on therapy to administration of metformin and insulin glargine.

6. The method according to claim 1, wherein the subject to be treated is an adult subject.

7. The method according to claim 1, wherein diabetes mellitus type 2 is not adequately controlled with metformin and insulin alone.

8. The method according to claim 7, wherein treatment with a dose of at least 1.5 g/day metformin and of at least 10 units/day of insulin alone for three months does not adequately control diabetes mellitus type 2.

9. The method according to claim 1, wherein the subject to be treated has a HbA1c value in the range of 7% to 10%, a fasting plasma glucose concentration of at least 7 mmol/L, or 2 hours postprandial plasma glucose of at least 11.1 mmol/L.

10. A pharmaceutical combination comprising
(a) desPro³⁶Exendin-4(1-39)-Lys₆-NH₂ or a pharmaceutically acceptable salt thereof,
(b) insulin glargine or a pharmaceutically acceptable salt thereof, and
(c) metformin or a pharmaceutically acceptable salt thereof.

11. The pharmaceutical combination according to claim 10, wherein the pharmaceutical combination is for treatment of diabetes mellitus type 2.
